(19) United States
(12) Patent Application Publication
(10) Pub. No.: US 2021/0366573 A1
(43) Pub. Date: Nov. 25, 2021

(54) VISUALIZATION AND SIMULATION OF GENOMES

(71) Applicant: King Abdullah University of Science and Technology, Thuwal (SA)

(72) Inventors: Azza Thamer Althagafi, Thuwal (SA); Robert Hoehndorf, Thuwal (SA)

(21) Appl. No.: 17/273,619

(22) PCT Filed: Sep. 4, 2019
(86) PCT No.: PCT/IB2019/057454

§ 371 Date: Mar. 4, 2021

Related U.S. Application Data
(60) Provisional application No. 62/726,546, filed on Sep. 4, 2018.

(51) Int. Cl. G16B 20/40; G16B 50/30; G16B 50/10; H04W 4/80

(52) U.S. Cl. CPC G16B 20/40; H04W 4/80; G16B 50/10; G16B 50/30

(57) ABSTRACT

Described is a computer-implemented method (CIM) for simulating a population of offspring genomes from the genomes of the two individuals, while taking into account linkage disequilibrium. The CIM annotates each genome in the population of offspring genomes with disease variants from genomic databases that contain variant information on diseases that involve more than one gene, and predicts pathogenic variants in the annotated population of offspring genomes. The CIM performs a statistical analysis of the annotated population of offspring genomes to determine the probability of morbidity within the simulated offspring population and displays the results on a user interface for visualization.

### CROSS-REFERENCE TO RELATED APPLICATIONS

This application claims benefit of U.S. Provisional Application No. 62/726,546 filed Sep. 4, 2018, which is incorporated herein by reference in its entirety.

### FIELD OF THE INVENTION

This invention is generally related to simulation, visualization, and interpretation of genomic data, particularly a computer-implemented method for simulating a population of a pair of individuals' offspring taking into account linkage disequilibrium, analyzing the simulated population to determine the likelihood of disease(s) associated with the population of offspring, and visualizing the results.

### BACKGROUND OF THE INVENTION

The contribution of genetics in human diseases ranges from almost 100% for monogenic (Mendelian) disorders to a much smaller percentage for complex diseases, including infectious disease (Hyman, Bulletin of the World Health Organization 2000, 78, 455-463). Understanding how variation(s) in an individual's genome relate to disease risk could help to prevent and/or predict health effects in individuals, generate better diagnoses and prognoses of disease, and investigate new approaches for treatment and development of new drugs (Bloss, et al., Psychiatric Clinics 2011, 34(1), 147-166). Studies in predicting health effects from genome sequences involve the interpretation of whole-exome sequencing (WES) or whole-genome sequencing (WGS) data of an individual(s), to identify causal mutations that could lead to an abnormal phenotype or a disease (Krier, et al., Dialogues in Clinical Neuroscience 2016, 18(3), 299). However, predicting possible health effects from genome sequences is an emerging discipline, and analysis and interpretation of variants in WGS or WES data remain a significant challenge due to the complex and large scale nature of these types of data (Krier, et al., Dialogues in Clinical Neuroscience 2016, 18(3), 299). Accordingly, a need exists to develop new and useful ways to enhance the understanding of how different variations of an individual's genome relates to diseases.

Another challenge in the analysis and interpretation of genomic data involves determining the risk of disease variants amongst offspring from two individuals. In the case of premarital testing, it can be advantageous to know from an early timeframe the different diseases or symptoms to which an offspring is susceptible and/or can inherit, based on wide variety of known genetically based diseases in infants. Several public databases are available, which contain information about the likely phenotypic effects of variants, including their penetrance and effect sizes (Trujillano, et al. Molecular Genetics & Genomic Medicine 2017, 5(1), 66-75). Several methods have also been developed to predict the likely phenotypic effects of variants using a wide range of features, namely evolutionary conservation, protein structure and function, network connectivity, and likely association of a variant and a phenotype (Trujillano, et al. Molecular Genetics & Genomic Medicine 2017, 5(1), 66-75). These methods provide information that can be employed to interpret individual genomes and are largely limited to Mendelian diseases. However, while Mendelian diseases may be predictable in offspring based on the genome sequences of parents using Mendel's laws of inheritance, this is not the case for more complex diseases including digenic, oligogenic, and multigenic diseases whose inheritance is affected by linkage disequilibrium that results in a non-uniform distribution of recombination centered around recombination hotspots. No method of generating genome data for a population of offspring while taking linkage disequilibrium of different alleles into account, and analyzing this phenomenon on inheritance of diseases has been previously described. Therefore, the development of methods and/or systems that can predict the probability of morbidity in a population while considering real-life phenomena remains an unmet need, and is an area of active research.

Therefore, it is an object of the invention to provide a computer-implemented method and/or system that predicts the probability of morbidity in a population of offspring genomes generated while taking into account linkage disequilibrium.

It is also an object of the invention to provide a computer-implemented method and/or system that visualizes the probability of morbidity in a population of offspring genomes generated while taking into account linkage disequilibrium.

It is a further object of the invention to provide a computer-implemented method and/or system that visualizes the genome of an individual, which has been annotated with one or more disease variants.

### SUMMARY OF THE INVENTION

A computer-implemented method (CIM) that allows a user to simulate a population of offspring from two individuals, by generating a population of offspring genomes from the genomes of the two individuals, while taking into account linkage disequilibrium has been developed. The CIM takes an input, a variant call format (VCF) file containing WGS or WES. The CIM annotates each genome in the population of offspring genomes with disease variants from one or more genomic databases that contain variant information on diseases that involve more than one gene. Further, the CIM predicts pathogenic variants in the annotated population of offspring genomes using the Mendelian Clinically Applicable Pathogenicity (M-CAP) score.

The CIM can also perform a statistical analysis of the annotated population of offspring genomes to determine the probability of morbidity or the likelihood of the occurrence of one or more diseases within the simulated offspring population and displays the results on a user interface for visualization and interpretation using chromosome ideograms.

The CIM can be utilized to visualize a single individual's genome or the probability of morbidity amongst a population of offspring genomes. The CIM can be utilized to analyze WES or WGS from all populations or regions of the world, and is particularly useful in regions of the world where consanguineous marriage is common.

Disclosed are computer-implemented methods (CIM) for analyzing genomic data. Generally, the CIM comprises: (a) generating a population of offspring genomes from the genomes of the two individuals, taking into account linkage disequilibrium; and (b) visualizing a probability of morbidity associated with the population of offspring genomes.

In some forms, the linkage disequilibrium taken into account can comprise the linkage disequilibrium found in a human population. In some forms, the genomes of the two individuals can be in a file format selected from the group consisting of a Variant Call Format (VCF), a Genome Variation Format (GVF), a Generic Feature Format (GFF), Gene Transfer Format (GTF), Tab Separated File (TSV), annovar file format, and masterVar file format, preferably VCF or GFF.

In some forms, the CIM can further comprise combining the genomes of the two individuals into a single file prior to step (a). In some forms, the CIM can further comprise a step of (i) annotating each genome in the population of offspring genomes with disease variants from one or more genomic databases after step (a) and prior to step (b). In some forms, the CIM can further comprise a step of (ii) predicting pathogenic variants in each genome in the population of offspring genomes after step (i) and prior to step (b). In some forms, the CIM can further comprise a step of (iii) performing a statistical analysis to determine the probability of morbidity after step (ii) and prior to step (b).

In some forms, predicting pathogenic variants can be performed using a Mendelian Clinically Applicable Pathogenicity (M-CAP) score, ClinPred, xgboost, cforest, VEST3, MetaSVM, REVEL, MetaLR, Eigen, GenoCanyon, REVEL, Fathmm_MKL, SIFT, Polyphen-2, or CADD, preferably M-CAP.

In some forms, taking into account linkage disequilibrium can comprise using recombination probabilities and a parameter that determines number of cross-overs per chromosome. In some forms, the recombination probabilities can be determined using a set of precomputed rate maps for a human genome build such as human genome build 37 or later versions such as GRCh38 and GRCh39.

In some forms, the one or more genomic databases are selected from the group consisting of ClinVar database, Genome-Wide Association Studies (GWAS) database, DIgenic disease DAtabase (DIDA), Pharmacogenomics Knowledgebase (PharmGKB), and combinations thereof. In some forms, the one or more genomic databases are the ClinVar database, GWAS database, DIDA, and PharmGKB. In some forms, the one or more genomic databases comprise a database containing information about Mendelian diseases; genetic associations for risk factors and/or complex diseases variants; digenic disease variants; oligogenic disease variants; pharmacogenomic variants; lifestyle factors; environmental factors; or a combination thereof. In some forms, the one or more genomic databases comprise a database containing information about complex diseases variants, digenic disease variants, oligogenic disease variants, or a combination thereof. In some forms, the one or more genomic databases are dynamic. In some forms, the one or more genomic databases are stored on one or more hardware modules.

In some forms, the genomes of the two individuals are provided to a first user interface hardware module, such as a graphical user interface (such as a digital screen), configured to receive genome from at least one of the two individuals, preferably wherein the first user interface hardware module is operably linked to the one or more hardware modules via ethernet, bluetooth, near field communication, WiFi, integrated circuits, or a combination thereof.

In some forms, visualizing the probability of morbidity occurs on the first user interface hardware module, a second user interface hardware module such as a graphical user interface (such as a digital screen), or both, preferably wherein the second user interface hardware module is operably linked to the one or more hardware modules ethernet, bluetooth, near field communication, WiFi, integrated circuits, or a combination thereof. In some forms, the second user interface hardware module is operably linked to the one or more hardware modules via ethernet, bluetooth, near field communication, WiFi, integrated circuits, or a combination thereof.

In some forms, generating the population of offspring genomes occurs on a third hardware module, preferably wherein the third hardware module is operably linked to:

the first user interface hardware module;
the second user interface hardware module; and/or
the one or more hardware modules.

In some forms, the first user interface hardware module or the second user interface hardware module and the third hardware module are on the same device or on different devices.

In some forms, the CIM further comprises a step of utilizing the probability of morbidity to counsel at least one of the two individuals.

In some forms, the CIM further comprises generating the population of offspring genomes over a number of generations/cycles such that the linkage disequilibrium of the population of offspring genomes last generated is comparable to the linkage disequilibrium found in the human population in which the linkage disequilibrium taken into account was found.

In some forms, generating the population of offspring comprises using recombination probabilities and a parameter that determines number of cross-overs per chromosome.

Also disclosed are computer-implemented systems (CIS) for analyzing gene expression data. Generally, the CIS comprises an informatics tool that generates a population of offspring genomes from the genomic information taking into account linkage disequilibrium, and provides processed results to a user.

In some forms, the CIS further comprises a user interface hardware module, such as a graphical user interface (such as a digital screen), configured to receive genomic information from the user or another user. In some forms, the CIS allows for implementation of any of the disclosed CIMs.

### BRIEF DESCRIPTION OF THE DRAWINGS

FIGS. 1A and 1B are schematics showing the overall workflow of the computer-implemented method. FIG. 1A shows the workflow for analyzing an individual's genome, and also for simulating a population of offspring genomes from two parent genomes to determine the probability of morbidity amongst the offspring. FIG. 1B shows the analysis of an individual's genome and also includes tools and databases involved.

FIG. 2 shows an individual's genome represented as an ideogram that has been annotated with variant information from a variety of databases, as well as a prediction score of disease variants.

FIG. 3 shows an ideogram that has been annotated with the probabilities of diseases associated with a population of simulated offspring genomes.

FIG. 4 is a line graph showing benchmark determinations of the length of time to generate sizes of populations of offspring genome.

FIG. 5 is a line graph showing the correlation between linkage disequilibrium for a population of simulated offspring genomes and a human population.

### DETAILED DESCRIPTION OF THE INVENTION

### I. Definitions

“Annotation,” “annotate,” or related terms, refer to the process of adding layers of analysis and interpretation to a DNA sequence (WGS or WES), in order to provide a biological significance to the entire DNA sequence of sections of the sequence. Annotation can be structural (involving the localization of gene elements), functional (associating a biological function to a gene), or both.

“Complex disease” refers to a disease whose causation can be associated with a mutation in at least two genes, such as digenic (two genes), oligogenic (between three and ten genes, inclusive), and/or polygenic (eleven or more genes) diseases. A disease is considered a complex disease when it is a disease that is multifactorial and may, for example, be associated with many variants each of which modifies disease risk. Multifactorial means that the disease can involve multiple genes, and optionally in combination with an individual's lifestyle (such as eating, exercising, drinking, smoking, etc.) and/or environmental factors.

“Database” refers to a repository that contains retrievable information. The database can be structured or non-structured, and is typically “dynamic.” “Dynamic” as relates to a database, refers to a database whose contents can change or be updated over time.

“Generation” or “cycle,” as relates to generating a population of offspring genomes refers to the number of iterations of randomly selecting and pairing the genomes of two offspring genomes from a population of offspring genomes, and further generating another population of offspring genomes from the randomly selected pair of genomes. The population can be of the same size over each iteration. In the context of the action of generating something (e.g., a population of offspring genomes), “generation” refers to the act of generating that something.

“Linkage disequilibrium” refers to the non-random association of alleles at two or more loci in a general population. When alleles are in linkage disequilibrium, haplotypes do not occur at the expected frequencies.

“Linkage map” refers to a representation of the linkage of genes in a chromosome, showing the relative positions of genes on a chromosome based on the frequencies with which genes are inherited together.

“Pathogenic variant” and “disease variant” are used interchangeably, and refer to a genetic alteration that enhances an individual's probability to develop or carry a particular disease or disorder.

### II. Computer-Implemented Methods

A computer-implemented method (CIM) that is not limited to any particular hardware of operating system is provided for processing and/or analyzing genomic data. The CIM allows a user to simulate a population of offspring from two individuals, by generating a population of offspring genomes from the genomes of the two individuals, while taking into account linkage disequilibrium. Preferably, the input data files to the CIM contain genomic data such as chromosome number (#CHROM), position (POS), reference alleles (REF) and alternate alleles (ALT), and information (INFO). A preferred file format for providing the genomic data of individuals is the variant call format (VCF). Preferably, the VCF file includes the chromosome number (#CHROM), position (POS), reference alleles (REF) and alternate alleles (ALT), and information (INFO). In the context of the disclosed CIM, “genome” refers to data that represents the genes and alleles of an individual, whether real (as for, e.g., the parents) or generated (as for, e.g., generated child or offspring genomes).

Following generation of a desirable population of offspring genomes, the CIM can annotate each genome in the population of offspring genomes with disease variants from one or more genomic databases. Preferred databases for annotating the population of offspring genomes include databases that contain variant information on diseases that involve more than one gene such as Genome-Wide Association Studies (GWAS) (MacArthur, et al., Nucleic Acids Research 2016, 45(D1), D896-D901) for genetic associations for risk factors and multigenic diseases, DIgenic diseases DAtabase (DIDA) (Gazzo, Nucleic Acids Research 2015, 44(D1), D900-D907) for the digenic disease variants (oligogenic inheritance), and Pharmacogenomics Knowledgebase (PharmGKB) for pharmacogenetic variants (Whirl-Carrillo, Clinical Pharmacology & Therapeutics 2012, 92(4), 414-417). Linkage disequilibrium is not particularly important for assessing strict Mendelian (monogenic) disease variants. However, most diseases are complex with multiple variants, and it has been recognized that even some diseases previously considered Mendelian diseases could involve multiple variants (Badano and Katsanis, Nat. Rev. Genet. 2002, 3, 779-789). A reason that diseases are still being classified as “Mendelian” arises from the fact that the majority of the phenotype can be ascribed to variations at a single locus (Badano and Katsanis, Nat. Rev. Genet. 2002, 3, 779-789). Accordingly, the genomic databases can also include Clinvar (Landrum, et al., Nucleic Acids Research 2013, 42(D1) D980-D985) for information about “Mendelian” diseases. The CIM further predicts pathogenic variants in the annotated population of offspring genomes. A preferred method for predicting pathogenic variants is the Mendelian Clinically Applicable Pathogenicity (M-CAP) score (Jagadeesh, et al., Nature Genetics 2016, 48(12), 1581).

The CIM can also perform a statistical analysis of the annotated population of offspring genomes to determine the probability of morbidity or the likelihood of the occurrence of one or more diseases within the simulated offspring population. Preferably, the CIM includes a user interface to facilitate implementation of and/or navigation throughout the CIM. For instance, the user interface facilitates user input of genomic data, execution of queries, and retrieval and analysis of results. The CIM can display an annotated genome for visualization and/or preferably display the determined probability of morbidity on a user interface in an appropriate manner. In some forms, the CIM can receive genomic data from an individual, annotate the genome by referencing one or more of the ClinVar, DIDA, GWAS, and PharmGKB databases, and predict disease variants using the M-CAP score.

Whether the CIM generates a population of offspring genome for analysis or analyzes the genomic of a single individual, visualization of the results can be based on a chromosomal ideogram that shows chromosomal positions at which functional variants have been found. Although any suitable user interface can be used, preferably, the user interface is a graphical user interface, such as one that is browser-enabled, i.e., a web-based application.

In some forms, the CIM can be performed via a browser-based application. A preferred browser-based application is termed Visualization and Simulation (VSIM). VSIM can be provided to a user, as source code and as a container at internet site github.com/bio-ontology-research-group/VSIM. “A container” refers to a standalone, executable package of software that includes everything needed to run an application: code, runtime, system tools, system libraries, and appropriate settings.

The CIM can be utilized to interpret and/or visualize a variety of genomic data, such as disease-causing variants in individual WGS or WES sequences. Given a WGS or WES from a pair of individuals as input, the CIM can simulate a cohort or population of offspring genomes, taking into account linkage disequilibrium by including the recombination probabilities and, preferably, a parameter that determines the number of cross-overs per chromosome. In some forms, the recombination probabilities are calculated from a set of precomputed rate maps following, as a non-limited example, the methods described in Su, et al., Science 1999, 286(5443), 1351-1353. The precomputed rate maps can be from a mammal genome build, preferably a human genome build such as human genome build 37 (GRCh37) or later versions including, but not limited to, GRCh38 and GRCh39. Variant information about members of this population of offspring genomes can be used to determine the probabilities that offspring of the two individuals from which the original WGS or WES were obtained would carry a disease, or develop a certain disease or phenotype. Therefore, not only can the CIM be used to interpret and visually explore individual genome sequences, it can also be used to perform premarital genetic testing.

Preferably, the CIM includes a classifier for predicting variants (such as disease) given a genome-containing file. Exemplary classifiers include M-CAP, ClinPred, xgboost, cforest, VEST3, MetaSVM, REVEL, MetaLR, Eigen, GenoCanyon, REVEL, or Fathmm_MKL described in Alirezaie, et al., American Journal of Human Genetics 2018, 103, 474-483, the contents of which are hereby incorporated by reference; SIFT (Ng and Henikoff, Nucleic Acids Research 2003, 31(13), 3812-3814); Polyphen-2 (Adzhubei, et al., Nature Methods 2010, 7(4), 248); or CADD (Kircher, et al., Nature Genetics 2014, 46(3), 310)). In some forms, the CIM includes the M-CAP, ClinPred, xgboost, or cforest score for predicting variants. Preferably, the CIM includes the M-CAP score that combines the pathogenicity scores of several other tools (including SIFT, Polyphen-2, and CADD).

In some forms, output from the CIM uses chromosome ideograms (Weitz, F1000Research 2017, 6). In general, chromosome ideograms are easy to interpret and include additional information about the variant and its likely phenotypic effect, making the CIM a user-friendly tool for visualization and interpretation of personal genomics data or data from a population of offspring genomes.

Preferably, the CIM described herein run on a computer-implemented system (CIS) capable of analyzing gene expression data. The CIS an informatics tool (such as VSIM) that generates a population of offspring genomes from the genomic information taking into account linkage disequilibrium, and provides processed results to a user. The CIS can include a user interface hardware module, such as a graphical user interface (such as a digital screen), configured to receive genomic information from the user or another user.

A. Data Files Containing Genomic Information

The CIM can receive WGS or WES from one or more input data files. Preferably, the input data files contain information about chromosome number (#CHROM), chromosome position (POS), reference alleles (REF) and alternate alleles (ALT), and information (INFO). The file format can be a Variant Call Format (VCF), a Genome Variation Format (GVF), a Generic Feature Format (GFF), Gene Transfer Format (GTF), Tab Separated File (TSV) such as BED, annovar file format, and masterVar file format. In some forms, the input file form is a VCF, preferably containing at minimum information about chromosome number (#CHROM), position (POS), reference alleles (REF) and alternate alleles (ALT), and information (INFO). The input data file can be dynamic, i.e., its contents can change or be updated over time. Therefore, in some forms, the input data file can be accessed from remote site or server where data in the files can be regularly updated. In some forms, a user can download an input data file and update data in the files to include a desirable data entry, such as zygosity of a disease variant, genomic function, the gene being affected, the transcript being affected, functional role of the coding variant, the nucleotide change in the transcript, the amino acid change in the protein, an individual's lifestyle (such as eating, exercising, drinking, smoking, etc.), the population of origin of the individual (e.g., the population from which one or both parents originate), the pedigree of an individual(s) (e.g. family of one or both parents), and/or environmental factors. Population information can be used to determine population-specific risk sites (such as from GWAS), while pedigree can be used to determine phenotypes associated with family members that share a haplotype with the individual.

B. Databases for Annotating Genomic Information

The CIM identifies candidate diseases variants by referencing to one or more databases. Preferably, these are genomic databases that contain information on genetic variation(s). The CIM can reference one database. Preferably, the CIM references at least two databases.

The databases can be dynamic Therefore, in some forms, the databases can be accessed from remote site or server where the databases are updated with new information, as needed. The databases (such as genomic databases) can be stored on one or more hardware modules locally or on a remote server. Preferably, the one or more hardware modules are operably linked with each other and/or to a user interface hardware module that receives input from a user. The link can be via ethernet, bluetooth, near field communication, WiFi, integrated circuits, or a combination thereof.

In some forms, a user can download a database and update data in the database to include a desirable data entry. Additional data that can be included in databases include, but are not limited to, zygosity of a disease variant, genomic function, the gene being affected, the transcript being affected, functional role of the coding variant, the nucleotide change in the transcript), the amino acid change in the protein, an individual's lifestyle (such as eating, exercising, drinking, smoking, etc.) and/or environmental factors. In some forms, these additional data exist in separate databases and, preferably, can be used to annotate genomic information. Exemplary databases include, but are not limited to: Clinvar (Landrum, et al., Nucleic Acids Research 2013, 42(D1) D980-D985) for information about Mendelian diseases, Genome-Wide Association Studies (GWAS) (MacArthur, et al., Nucleic Acids Research 2016, 45(D1), D896-D901) for genetic associations for risk factors and multigenic diseases, DIgenic diseases DAtabase (DIDA) (Gazzo, Nucleic Acids Research 2015, 44(D1), D900-D907) for the digenic disease variants (oligogenic inheritance), Pharmacogenomics Knowledgebase (PharmGKB) for pharmacogenetic variants (Whirl-Carrillo, Clinical Pharmacology & Therapeutics 2012, 92(4), 414-417), Exome Aggregation Consortium (ExAC), and 1000 Genomes.

In some forms, the CIM references ClinVar. In some forms, the CIM references GWAS. In some forms, the CIM references DIDA. In some forms, the CIM references PharmGKB. In some forms, the CIM references ExAC. In some forms, the CIM references 1000 Genomes. In some forms, the CIM references ClinVar and GWAS. In some forms, the CIM references ClinVar and DIDA. In some forms, the CIM references ClinVar and PharmGKB. In some forms, the CIM references ClinVar and ExAC. In some forms, the CIM references ClinVar and 1000 Genomes. In some forms, the CIM references DIDA and PharmGKB. In some forms, the CIM references DIDA and ExAC. In some forms, the CIM references DIDA and 1000 Genomes. In some forms, the CIM references ClinVar, DIDA, and GWAS. In some forms, the CIM references ClinVar, GWAS, and PharmGKB. In some forms, the CIM references ClinVar, DIDA, and PharmGKB. In some forms, the CIM references ClinVar, DIDA, and ExAC. In some forms, the CIM references ClinVar, DIDA, and 1000 Genomes. In some forms, the CIM references ClinVar, GWAS, DIDA, and PharmGKB. In some forms, the CIM references ClinVar, GWAS, DIDA, and ExAC. In some forms, the CIM references ClinVar, GWAS, DIDA, and 1000 Genomes. In some forms, the CIM references ClinVar, GWAS, DIDA, PharmGKB, and ExAC. In some forms, the CIM references ClinVar, GWAS, DIDA, PharmGKB, and 1000 Genomes. In some forms, the CIM references ClinVar, GWAS, DIDA, PharmGKB, ExAC, and 1000 Genomes.

Preferably, prior to predicting variants, a tool can be used to annotate the WGS or WES in the input data files or the population of offspring genomes generated by the CIM. Preferably, the tool performs a functional annotation. Exemplary annotation tools include, but are not limited to, ANNOVAR tool (internet site annovar.openbioinformatics.org/en/latest/(accessed: 2018-1-1)), SnpEff (internet site snpeff.sourceforge.net (accessed: 2019-08-13)), SnpSift (internet site snpeff.sourceforge.net (accessed: 2019-08-13)), ClinEff (web site dnaminer corn (accessed: 2019-08-13)), VEP (McLaren, et al., Bioinformatics 2010; 26(16):2069-70), VAAST (Hu, et al., Genet Epidemiol. 2013, 37(6):622-34), AnnTools (Makarov, et al., Bioinformatics 2012, 28(5):724-5), and vcfanno (Pederson, et al., Genome Biology 2016, 17:118). In some forms, the annotation tool is ANNOVAR.

C. Analysis

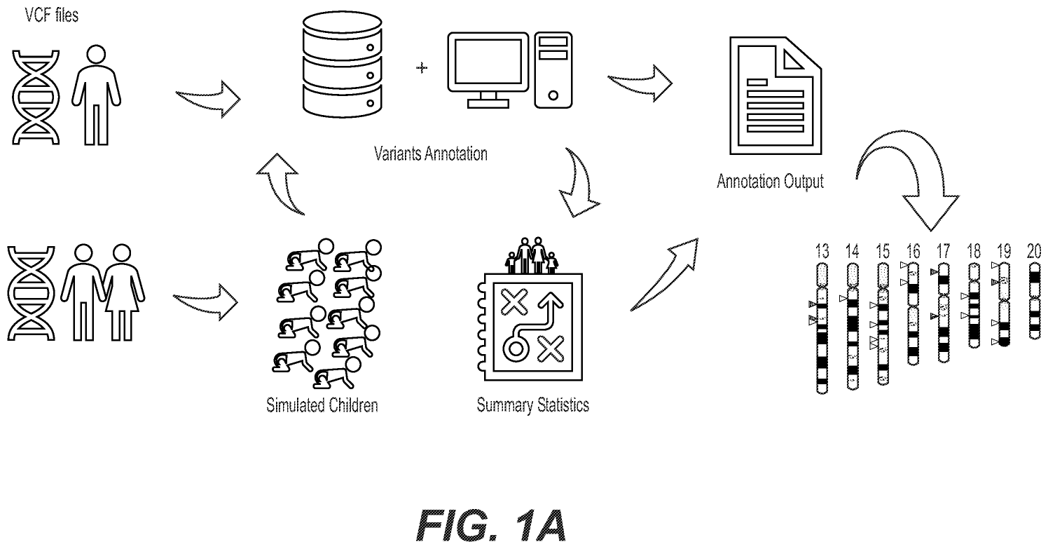

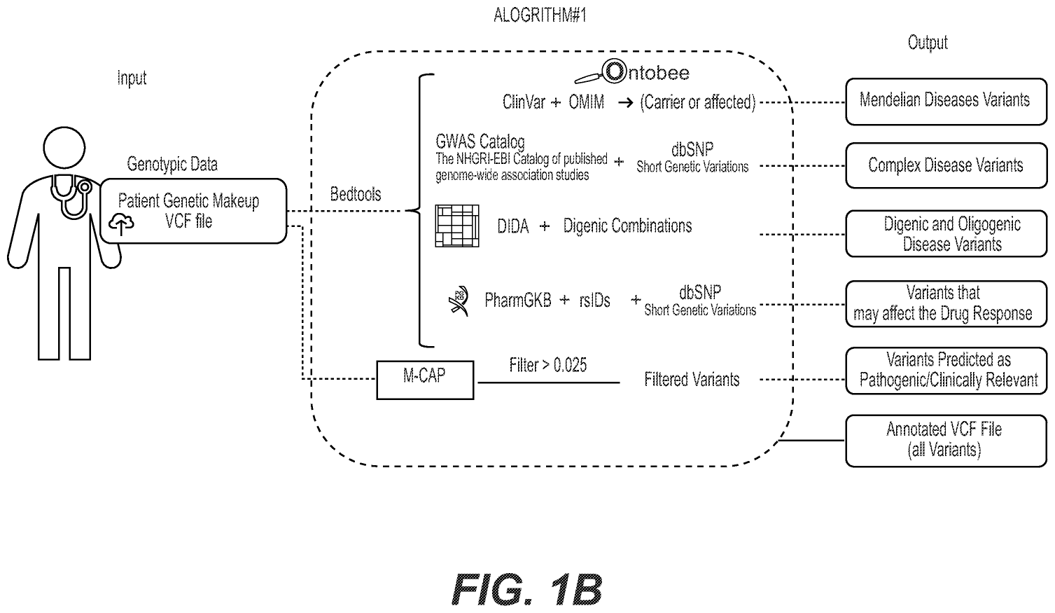

FIGS. 1A and 1B provide overviews of the overall workflow. The genome to be analyzed can be provided to the CIM in any of the file formats described above: a GVF, a GFF, a GTF, a TSV such as BED, an annovar file format, and masterVar file format, preferably as a VCF. Preferably, the genome is provided via a first user interface hardware module including, but not limited to, a graphical user interface (such as a digital screen) configured to receive the genome of an individual. This individual can be from at least one of two individuals whose genome will be used to generate a population of offspring genomes. Preferably, the first user interface hardware module is operably linked to the one or more hardware modules. The one or more hardware modules can be a processor (such as a computer processing unit) that runs one or more processes of the CIM, or a storage device (local or remote server) containing genome database. The link can be via ethernet, bluetooth, near field communication, WiFi, integrated circuits, or a combination thereof.

The input data file containing the genome or each CIM-generated offspring genome can be annotated by referencing any of the genome databases described above: ClinVar, GWAS, DIDA, PharmGKB, ExAC, and 1000 Genomes, preferably ClinVar, GWAS, DIDA, and PharmGKB. Where the analysis involves visualizing an individual's genome, the first user interface receives an input file containing the genome of that individual and the CIM directly annotates that file. For predicting the probability of morbidity in a population of offspring genomes, the simulator takes two files as input, optionally combines them into a single, and generates a population of offspring genomes taking into account the recombination probabilities as described herein.

Preferably, the CIM further predicts pathogenic variants for the annotated individual genome or for each genome amongst the population of offspring genomes using a classifier, such as M-CAP, ClinPred, xgboost, cforest, VEST3, MetaSVM, REVEL, MetaLR, Eigen, GenoCanyon, REVEL, Fathmm_MKL, SIFT, Polyphen-2, or CADD, preferably M-CAP. Optionally, the classifier further identifies all the associated diseases in the genome by utilizing databases referenced by the CIM. Preferably, the classifier assigns a score for each variant in the annotated genome. In some forms, this likelihood score mis-classifies no more than 5% of pathogenic variants, while reducing variants of uncertain significance (Jagadeesh, et al., Nature Genetics 2016, 48(12), 1581). In some forms, the computed scores can be directly used by clinicians to interpret variants of an uncertain consequence (Jagadeesh, et al., Nature Genetics 2016, 48(12), 1581).

In the case of a simulated population of offspring genomes, the CIM can also perform a statistical summary (such as likelihoods or probabilities) for all the diseases associated with the population of offspring genomes generated. Finally, a new file is generated, containing annotations of all the related information in a format that can be visualized.

The final output file can be in a format that can be visualized via an interface. The final output file can be an open-standard file format that is computer-programming language independent. The final output file can also be human-readable. In some forms, the file contains a collection of attribute-value pairs, and preferably an ordered list of values. In some forms, the final output file is a JavaScript Objection Notation (JSON) file. Genomes and chromosomes are often represented visually through the use of an ideogram, i.e., a schematic representation of chromosomes, which preferably shows the relative size of the chromosomes and their characteristic patterns. While ideograms may appear simplistic, they greatly facilitate analysis of genomic data. The Ideogram.js annotation sets were used (“Ideogram weitz em,ideogram [online]0.2015,” internet site github.com/eweitz/ideogram (accessed: 1 Sep. 2018)) for chromosome visualization, and for overlaying the visual representation of each chromosome with the information obtained from annotating variants in a VCF file. Ideogram supports drawing and animating genome-wide datasets.

In some forms, visualizing the results (such as annotated the genomes or probability of morbidity) occurs on a user interface hardware module (that can be the same as the first user interface hardware module configured to receive the genome of an individual as input), a second user interface hardware module such as a graphical user interface (such as a digital screen), or both. Preferably, the second user interface hardware module is operably linked to one or more hardware modules. The one or more hardware modules can be a processor (such as a computer processing unit) that runs one or more processes of the CIM, or a storage device (local or remote server) containing genome database. Preferably, the one or more hardware modules include a processor (such as a computer processing unit) that runs one or more processes of the CIM. The link can be via ethernet, bluetooth, near field communication, WiFi, integrated circuits, or a combination thereof. In some forms, generating the population of offspring genomes occurs on a third hardware module such as a processor (including computer processing unit) that runs one or more processes of the CIM. Preferably, the third hardware module is operably linked to the first user interface hardware module; the second user interface hardware module; and/or the one or more hardware modules. The link between the third hardware module, the first user interface hardware module, and the second user interface hardware module can be via ethernet, bluetooth, near field communication, WiFi, integrated circuits, or a combination thereof. In some forms, the first user interface hardware module or the second user interface hardware module and the third hardware module are on the same device or on different devices.

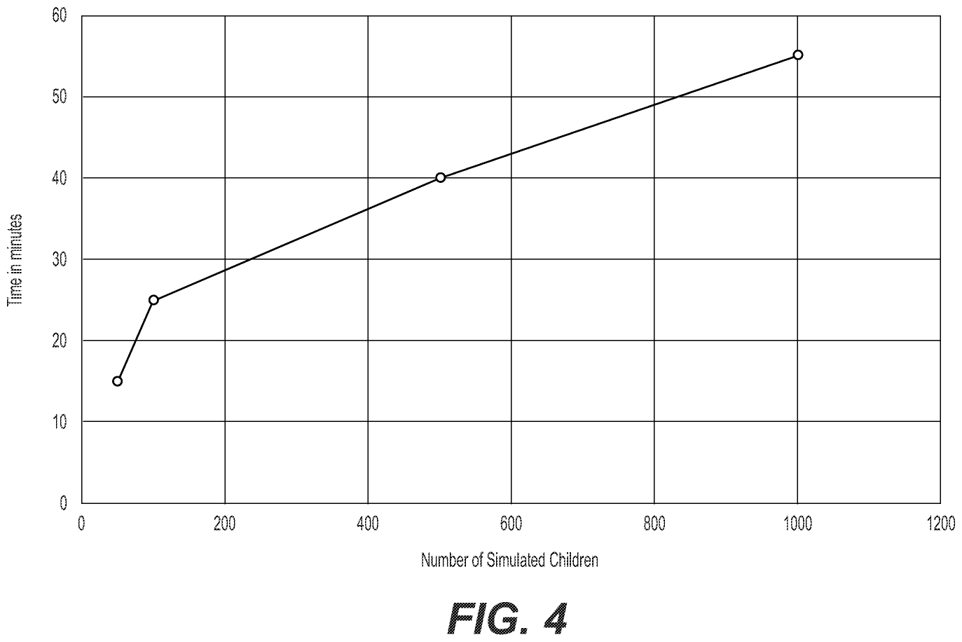

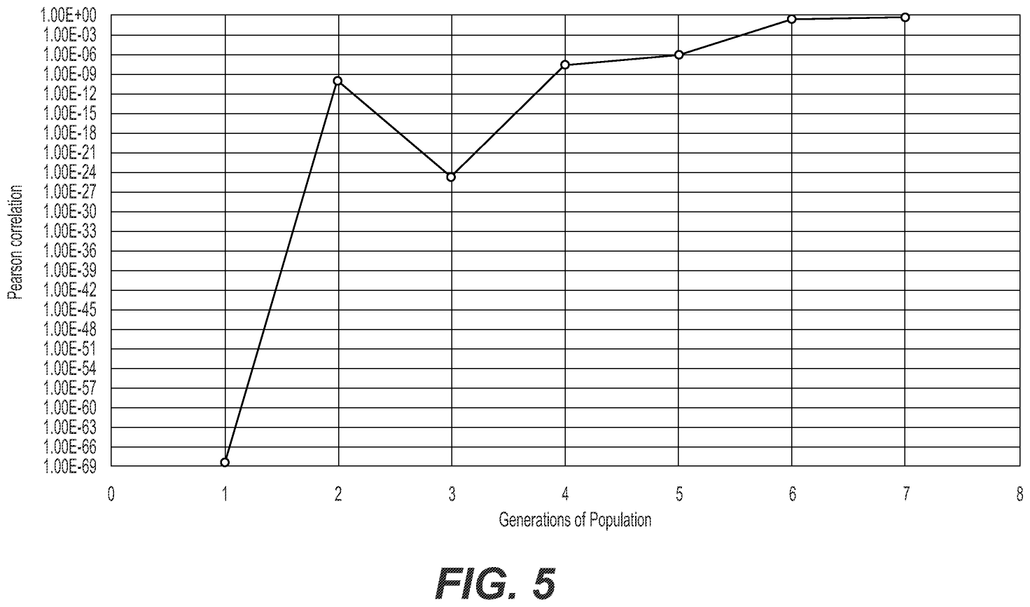

The performance of the CIM can be evaluated by determining how quickly it completes tasks. The time it takes to complete tasks when analyzing a single WGS or WES depends on the size of the input data file. For example, it can take approximately 10 minutes to generate a final output of a VCF containing three million SNPs, utilizing an Intel i7 processor at 2.5 GHz with 16 GB of memory. For determining the likelihood of offspring having or carrying a particular disease, the simulation time can depend on the size of the file and/or the number of offspring genomes to be generated. Referring to FIG. 4, the time to simulate a number of offspring increases linearly with the number of simulations to perform. Lastly, FIG. 5 shows that a strong correlation with linkage disequilibrium in a human population emerges after only a few generations, which validates the inclusion of linkage disequilibrium in the disclosed methods.

### III. Methods of Using

The described CIM can be utilized to analyze WES or WGS from all populations or regions of the world. The CIM can be utilized to visualize a single individual's genome or the probability of morbidity amongst a population of offspring genomes. The CIM is particularly relevant in regions of the world where consanguineous marriage is common as a result of socio-cultural factors including religion and ethnicity (Bener and Mohammad, Egyptian Journal of Medical Human Genetics 2017, 18(4), 315-320; Bener and Hussain, Paediatric and Perinatal Epidemiology 2006, 20(5), 372-378; Bener, et al., QNRS Repository 2011, 2011(1), 1657; Modell and Dan, Nature Reviews Genetics 2002, 3(3), 225).

The disclosed compositions and methods can be further understood through the following numbered paragraphs.

1. A computer-implemented method (CIM) for analyzing genomic data, the CIM comprising:

(a) generating a population of offspring genomes from the genomes of the two individuals, taking into account linkage disequilibrium; and

(b) visualizing a probability of morbidity associated with the population of offspring genomes.

2. The CIM of paragraph 1, wherein the linkage disequilibrium taken into account comprises the linkage disequilibrium found in a human population.

3. The CIM of paragraph 1 or 2, wherein the genomes of the two individuals are provided in a file format selected from the group consisting of a Variant Call Format (VCF), a Genome Variation Format (GVF), a Generic Feature Format (GFF), Gene Transfer Format (GTF), Tab Separated File (TSV), annovar file format, and masterVar file format, preferably VCF or GFF.

4. The CIM of any one of paragraphs 1 to 3, further comprising combining the genomes of the two individuals into a single file prior to step (a).

5. The CIM of any one of paragraphs 1 to 4, further comprising a step of (i) annotating each genome in the population of offspring genomes with disease variants from one or more genomic databases after step (a) and prior to step (b).

6. The CIM of any one of paragraphs 1 to 5, further comprising a step of (ii) predicting pathogenic variants in each genome in the population of offspring genomes after step (i) and prior to step (b).

7. The CIM of paragraph 5 or 6, further comprising a step of (iii) performing a statistical analysis to determine the probability of morbidity after step (ii) and prior to step (b).

8. The CIM of paragraph 6 or 7, wherein predicting pathogenic variants is performed using a Mendelian Clinically Applicable Pathogenicity (M-CAP) score, ClinPred, xgboost, cforest, VEST3, MetaSVM, REVEL, MetaLR, Eigen, GenoCanyon, REVEL, Fathmm_MKL, SIFT, Polyphen-2, or CADD, preferably M-CAP.

9. The CIM of any one of paragraphs 1 to 8, wherein taking into account linkage disequilibrium comprises using recombination probabilities and a parameter that determines number of cross-overs per chromosome.

10. The CIM of paragraph 9, wherein the recombination probabilities are determined using a set of precomputed rate maps for a human genome build such as human genome build 37 or later versions such as GRCh38 and GRCh39.

11. The CIM of any one of paragraphs 5 to 10, wherein the one or more genomic databases are selected from the group consisting of ClinVar database, Genome-Wide Association Studies (GWAS) database, DIgenic disease DAtabase (DIDA), Pharmacogenomics Knowledgebase (PharmGKB), and combinations thereof.

12. The CIM of any one of paragraphs 5 to 11, wherein the one or more genomic databases are the ClinVar database, GWAS database, DIDA, and PharmGKB.

13. The CIM of any one of paragraphs 5 to 12, wherein the one or more genomic databases comprise a database containing information about Mendelian diseases; genetic associations for risk factors and/or complex diseases variants; digenic disease variants; oligogenic disease variants; pharmacogenomic variants; lifestyle factors; environmental factors; or a combination thereof.

14. The CIM of any one of paragraphs 5 to 13, wherein the one or more genomic databases comprise a database containing information about complex diseases variants, digenic disease variants, oligogenic disease variants, or a combination thereof.

15. The CIM of any one of paragraphs 5 to 14, wherein the one or more genomic databases are dynamic.

16. The CIM of any one of paragraphs 5 to 15, where the one or more genomic databases are stored on one or more hardware modules.

17. The CIM of any one of paragraphs 1 to 16, wherein the genomes of the two individuals are provided to a first user interface hardware module, such as a graphical user interface (such as a digital screen), configured to receive genome from at least one of the two individuals, preferably wherein the first user interface hardware module is operably linked to the one or more hardware modules via ethernet, bluetooth, near field communication, WiFi, integrated circuits, or a combination thereof.

18. The CIM of 17, wherein visualizing the probability of morbidity occurs on the first user interface hardware module, a second user interface hardware module such as a graphical user interface (such as a digital screen), or both, preferably wherein the second user interface hardware module is operably linked to the one or more hardware modules ethernet, bluetooth, near field communication, WiFi, integrated circuits, or a combination thereof.

19. The CIM of paragraph 18, wherein the second user interface hardware module is operably linked to the one or more hardware modules via ethernet, bluetooth, near field communication, WiFi, integrated circuits, or a combination thereof.

20. The CIM of any one of paragraphs 17 to 19, wherein generating the population of offspring genomes occurs on a third hardware module, preferably wherein the third hardware module is operably linked to:

the first user interface hardware module;
the second user interface hardware module; and/or
the one or more hardware modules.

21. The CIM of paragraph 18 or 19, wherein the first user interface hardware module or the second user interface hardware module and the third hardware module are on the same device or on different devices.

22. The CIM of any one of paragraphs 1 to 21, further comprising a step of utilizing the probability of morbidity to counsel at least one of the two individuals.

23. A computer-implemented system (CIS) for analyzing gene expression data, comprising an informatics tool that generates a population of offspring genomes from the genomic information taking into account linkage disequilibrium, and provides processed results to a user.

24. The CIS of paragraph 23, further comprising a user interface hardware module, such as a graphical user interface (such as a digital screen), configured to receive genomic information from the user or another user.

25. The CIS of paragraph 23 or 24, wherein the CIS allows for implementation of the CIM of any one of paragraphs 1 to 22.

26. The CIM of paragraph 2, further comprising generating the population of offspring genomes over a number of generations/cycles such that the linkage disequilibrium of the population of offspring genomes last generated is comparable to the linkage disequilibrium found in the human population in which the linkage disequilibrium taken into account was found.

27. The CIM of any one of paragraphs 1 to 8 or 26, wherein generating the population of offspring comprises using recombination probabilities and a parameter that determines number of cross-overs per chromosome.

### EXAMPLES

### Example 1: Visualization and Simulation (VSIM) of Genomes

Recent studies show that the prevalence of consanguineous marriages varies from 33% to 68% in different countries (Bener and Mohammad, Egyptian Journal of Medical Human Genetics 2017, 18(4), 315-320), and it is estimated at about 58% in Saudi Arabia (El Mouzan, et al., Annals of Saudi medicine 2008, 28(3), 169), 50% in United Arab Emirates (Bener, et al., Human Heredity 1996, 46(5), 256-264), 50% in Oman (Rajab and Patton, Annals of Human Biology 2000, 27(3), 321-326), and 68% in Egypt (Mokhtar and Abdel-Fattah, European Journal of Epidemiology 2001, 17(6), 559-565). As a response to the health effects arising from high rates of consanguinity, genetic testing before marriage has been introduced by government health authorities in some of these countries to identify individual who are carriers of autosomal recessive disorders or individuals who have a genetic predisposition that may produce a disease in their offspring (Bener and Mohammad, Egyptian Journal of Medical Human Genetics 2017, 18(4), 315-320; Bener and Hussain, Paediatric and Perinatal Epidemiology 2006, 20(5), 372-378; Bener, et al., QNRS Repository 2011, 2011(1), 1657).

Several studies have reported on the common effects of consanguineous marriage on health, and have focused on its impact on reproduction, rare Mendelian disorders, and childhood mortality (Bener and Mohammad, Egyptian Journal of Medical Human Genetics 2017, 18(4), 315-320; Bener and Hussain, Paediatric and Perinatal Epidemiology 2006, 20(5), 372-378; Bittles and Black, Proceedings of the National Academy of Sciences 2010, 107(suppl 1), 1779-1786; Bittles, et al., Annals of Human Biology 2002, 29(2), 111-130; Abdulrazzaq, et al., Clinical Genetics 1997, 51(3), 167-173; Cherkaoui, et al., International Journal of Anthropology 2005, 20(3-4), 199-206; Bittles, Developmental Medicine and Child Neurology 2003, 45(8), 571-576; Wright and Hastie, Genome Biology 2001, 2(8), comment2007.1-comment2007.8; Ben Arab, et al., Genetic Epidemiology: The Official Publication of the International Genetic Epidemiology Society 2004, 27(1), 74-79, 2004; Bener, et al., Cancer 2001, 92(1), 1-6; Pedersen, Public Health Genomics 2002, 5(3), 178-181). However, there is a lack of awareness about consanguinity diseases transmittable to an offspring, especially in Saudi Arabia (Ibrahim, et al., Journal of Infection and Public Health 2013, 6(1), 41-54), which causes a lot of heredity diseases giving rise to great suffering for the next generation of offspring. Therefore, for couples considering marriage, premarital screening helps to identify potential health problems and risks for themselves and also their offspring. For example, the Saudi Premarital Screening and Genetic Counseling (PMSGC) program named the “Healthy Marriage Program” is part of a national project led by the Saudi Ministry of Health (Alrajhi, Journal of Infection and Public Health 2009, 2(1), 4-6) to bring awareness to the premarital screening. There are comprehensive PMSGC program guidelines distributed to all workers in the program. Couples planning to get married are required to apply for premarital screening tests (Memish and Saeedi, Annals of Saudi Medicine 2011, 31(3), 229). These tests screen for specific diseases, in particular hemoglobinopathies such as sickle cell anemia and thalassemias, as well as infectious diseases such as human immunodeficiency virus, hepatitis B virus, and hepatitis C virus (Ibrahim, et al., Journal of Infection and Public Health 2011, 4(1), 30-40). While infectious disease screening is important, it does not address the problem of heritable diseases arising from consanguinity.

The analysis described below involve simulating populations of offspring genomes starting from two parents taking into account linkage disequilibrium and visualizing the results.

### Materials and Methods

The annotation algorithm for VSIM uses variant call format (VCF) files directly, which at minimum must include the chromosome number (#CHROM), position (POS), reference alleles (REF) and alternate alleles (ALT), and information (INFO). The variants are annotated with many different pieces of information related to the diseases, which provides additional information as described in more detail below. In this example, VSIM implements a user-friendly web interface that runs on an Apache web server. The communication between the client-side layer and the server-side takes place based on JavaScript.

A. Databases

Four databases were used for annotation. Table 1 provides high level information about each of the databases. The following is a general overview about each of them:

(i) ClinVar (Landrum, et al., Nucleic Acids Research 2013, 42(D1) D980-D985) is a database of gnomic variants and the interpretation of their relevance to diseases. It identifies the relationships among medically important variants and phenotypes. The variations contained in this database are in VCF format and ClinVar contains a mixture of variations asserted to be pathogenic as well as those known to be non-pathogenic (Landrum, et al., Nucleic Acids Research 2013, 42(D1) D980-D985), with regard to their clinical significance. However, this work focused on the pathogenic and likely pathogenic variants. Therefore, as a result of this restriction, 84,536 variants out of 396,647 Single nucleotide polymorphisms (SNPs) were obtained.

(ii) GWAS (MacArthur, et al., Nucleic Acids Research 2016, 45(D1), D896-D901) is a statistical method that determines the associations between SNPs and particular traits or disorders. It looks for many factors at once. This takes full advantage of all the SNPs and uses them as sign posts for different phenotypes or traits. The GWAS Catalog (MacArthur, Nucleic Acids Research 2016, 45(D1), D896-D901) now contains over 2500 unique SNP-trait associations, i.e., associations between single nucleotide variants and phenotypes or diseases. It has been very successful in terms of identifying locations in the genome that are associated with disease. The GWAS Catalog contains information about variants (in particular their genomic position) and an association with, usually, polygenic diseases. There are 69,460 variants from the GWAS Catalog, and all of them were used in the experiments.

(iii) DIDA (Gazzo, Nucleic Acids Research 2015, 44(D1), D900-D907) is a database that provides a detailed and/or comprehensive information on genes and associated genetic variants that are associated with digenic diseases. It includes 213 digenic combinations which composed of 364 distinct variants. This involved in 44 digenic diseases (Gazzo, Nucleic Acids Research 2015, 44(D1), D900-D907). From this database digenic inheritance which is the simplest form of the oligogenic inheritance for genetically complex diseases was investigated. Inheritance is digenic “when the variant genotypes at two loci explain the phenotypes of some patients and their unaffected (or more mildly affected) relatives more clearly than the genotypes at one locus alone” (Schïffer, Journal of Medical Genetics 2013, 50(10), 641-652), i.e., particular genotypes in exactly two genes explain the disease or phenotype in a patient. DIDA provides an opportunity to further focus on, and investigate, the digenic inheritance model. From this database information the variants were annotated with digenic diseases.

(iv) PharmGKB (Whirl-Carrillo, Clinical Pharmacology & Therapeutics 2012, 92(4), 414-417) investigates the association of genetic variation and a drug's efficiency. PharmGKB contains pharmacogenetic information related to 3,070 variants. From PharmGKB, variants were annotated with different drug responses.

TABLE 1
Databases used

| Databases | Purpose | Source | # Variants |
|---|---|---|---|
| ClinVar | Mendelian Diseases | NCBI | 84,536 |
| GWAS | Complex Diseases | GWAS Catalog | 69,460 |
| PharmGKB | Drug response | Pharmgkb.org | 3,070 |
| DIDA | Digenic diseases | DIDA | 464 |

B. M-CAP Pathogenicity Prediction

The development of the disease or symptoms of the disease is more likely to appear in the individual when such a variant (or mutation) is inherited. Mendelian Clinically Applicable Pathogenicity (M-CAP) (Jagadeesh, et al., Nature Genetics 2016, 48(12), 1581) is a pathogenicity predictor for rare missense variants within the human genome. It is the first pathogenicity classifier for rare missense variants in the human genome that is tuned to the high sensitivity required in clinical settings. It combines pathogenicity scores of several other tools (including SIFT (Ng and Henikoff, Nucleic Acids Research 2003, 31(13), 3812-3814), Polyphen-2 (Adzhubei, et al., Nature Methods 2010, 7(4), 248) and CADD (Kircher, et al., Nature Genetics 2014, 46(3), 310)) within a new machine learning model containing additional features. M-CAP was used to predict pathogenicity in all the variants in a VCF file. The ANNOVAR tool (internet site annovar.openbioinformatics.org/en/latest/(accessed: 2018-1-1)) was used to perform the annotations.

C. Simulation

The simulation was implemented based on the Real Time Genomics (RTG) simulation tool (Cleary, BioRxiv 2015, p. 023754). RTG provides a blueprint platform for genomic analysis. The RTG tools software is delivered as an executable file, to be run with multiple commands executed through a command line interface. RTG, amongst others, supports the generation of child genomes from two VCF files that represent parents, and contains parameters that allow for specifying the number of recombinations per chromosome and for adding random new mutations in children. However, RTG's simulation algorithm for generating offspring genomes only produces completely random recombination of input from VCF files. The present inventors realized that such random recombination does not reflect the reality of the combination and recombination of parental genomes. In particular, the present inventors noted that linkage disequilibrium produces non-random recombination of the loci and alleles of the parental genomes and that RTG does not have any capability to simulate populations while maintaining linkage disequilibrium (Cleary, BioRxiv 2015, p. 023754). Thus, the simulation of a child genome from out-of-the-box RTG is based on Mendelian inheritance principles only. The present inventors realized that, although such a Mendelian inheritance simulation would naturally produce some linkage disequilibrium due to genomic proximity of alleles, it would not simulate the many other factors that lead to varying levels of linkage dis-equilibrium (such as recombination hot spots or the many functional reasons for alleles being associated). The present inventors thus resolved to add simulation of real-world linkage disequilibrium into the generation of child genomes. Therefore, in this study, the RTG source code was reconfigured to capture linkage disequilibrium.

D. Visualization

Interpretation of genomics data is complex and challenging, especially due to the large-scale nature of these types of data. Presenting a description of genomics data in a visual format would allow most users to better analyze and appreciate content of the data. Genomes and chromosomes are often represented visually through the use of an ideogram, i.e., a schematic representation of chromosomes, which preferably shows the relative size of the chromosomes and their characteristic patterns. While ideograms may appear simplistic, they greatly facilitate analysis of genomic data. The Ideogram.js annotation sets were used (“Ideogram weitz em,ideogram [online]0.2015,” internet site github.com/eweitz/ideogram (accessed: 1 Sep. 2018)) for chromosome visualization, and for overlaying the visual representation of each chromosome with the information obtained from annotating variants in a VCF file. Ideogram supports drawing and animating genome-wide datasets. The formatted annotation data was then plugged into a lightly modified example from the Ideogram repository (“Ideogram weitz em,ideogram [online]0.2015,” internet site github.com/eweitz/ideogram (accessed: 1 Sep. 2018)) to provide an informative explanation regarding the diseases associated with a particular position in the chromosome. Ideogram.js uses JavaScript and Scalable Vector Graphics (SVG) to draw chromosomes and associated annotation data in HTML documents. It leverages D3.js, a popular JavaScript visualization library, for data binding, DOM manipulation, and animation (Bostock, et al., IEEE Transactions on Visualization & Computer Graphics 2011, 12, 2301-2309). By relying only on JavaScript libraries, HTML and CSS, Ideogram can function entirely in a web browser, with no server-side code required, which simplifies embedding ideograms in a web application.

After obtaining the information related to all the chromosome positions, the next step is to parse genomic features (chromosome name, annotation, start and stop of a coding region) and gene type (e.g. mRNA, ncRNA) from a generic feature format (GFF) file in the NCBI Homo sapiens Annotation Release, for instance NCBI human genome version 37. These data are combined and formatted into a compressed JSON structure file. This file, e.g. ID325476.json, represents the final output of the visualization data, and contains all the data used by the client-side in Ideogram.js.

E. Implementation

For testing VSIM, sequence data of human genomes obtained from the 1000 Genomes project (“Ideogram igsr: The international genome sample resource,” web site internationalgenome.org/data (accessed: 1 Sep. 2017)) were analyzed. Variants were listed in a VCF file (VCFv4.0) (Danecek, et al., Bioinformatics 2011, 27(15), 2156-2158). The VCF files have all the individual's information for each chromosome. By using Jvarkit (Lindenbaum, “Jvarkit: java-based utilities for bioinformatics,” FigShare, doi, vol. 10, p. m9, 2015) individual VCF files were extracted from the main VCF file for all the chromosomes, and then the chromosomes list for each individual was concatenated using VCFtool (Danecek, et al., Bioinformatics 2011, 27(15), 2156-2158).

The four databases, ClinVar, GWAS, DIDA, PharmGKB, were used for annotation (dated 30 Aug. 2018), using the reference genome of GRCh37 as the main genomic variants set. However, since some of the databases (ClinVar, GWAS Catalog, and DIDA) are regularly updated, variants were identified from these databases from a remote up-to-date server for the annotation.

For identifying the candidate diseases variants with ClinVar database, mode of inheritance (MOI) was downloaded for diseases in Online Mendelian Inheritance in Man (OMIM) from the human phenotype ontology (HPO) database (Köhler, et al., Nucleic Acids Res. 2016, 45(D1), D865-D876). As a result, a total of 6,843 MOI records were obtained, which were classified as ‘Dominant,’ Recessive,′ or ‘Others.’ Subsequently, the genotype (homo- or heterozygote) of a variant in a VCF file was used to annotate an individual's genome as affected by a disease or carrying (heterozygote) disease variant.

Moreover, along with MOI, the zygosity of a variant was used as a guide to decide whether a person will get a certain disease. Zygosity information is not provided in ClinVar, but rather in the given VCF file (Landrum, et al., Nucleic Acids Res. 2017, 46(D1), D1062-D1067). Zygosity is represented in the genotype (GT) field of the file. For example, in VCF file a heterozygous variant will have a genotype value 0/1, while the homozygous variant will have 1/1. The pathogenic variant disease was associated with a variant based on genotype information and MOI. For instance, for a specific variant, if the disease mode of inheritance is recessive, and the zygosity of the variant in VCF file is 0/1, then this person will carry the diseases associated with that variant, i.e., this individual is not infected with the disease but is a healthy carrier of the disease. And if the disease mode of inheritance is dominant, and the zygosity of the variant in VCF file is 0/1 or 1/1, then this person will have the diseases associated with that variant. As an example, the pathogenic variant rs1801265 in DPYD is associated with (OMIM:274270) this OMIM is recessive, and the genotype with this VCF file that match with the position of the rs1801265 is 0/1 or 1/1, then the person will carry this disease. On the other hand, pathogenic variant rs3214759 in CRYGB is associated with (OMIM:615188) this OMIM is dominant, and the genotype with this VCF file that match with the position of the rs1801265 is 1/1, then the person will have this disease.

However, in this example, zygosity information was only incorporated in the ClinVar database. The information for the remaining databases was retrieved by finding the exact match with the Chromosome, position, REF alleles and ALT alleles.

M-CAP score was used to assign a score for each variant in the input VCF file. This likelihood score aims to mis-classify no more than 5% of pathogenic variants, while reducing variants of uncertain significance (Jagadeesh, et al., Nature Genetics 2016, 48(12), 1581). M-CAP uses a gradient boosting tree that is a supervised learning classifier that outclasses other tools at analyzing the nonlinear interactions between features and has many state-of-art performance in different classification tasks (Jagadeesh, et al., Nature Genetics 2016, 48(12), 1581). The computed scores can be directly used by clinicians to interpret variants of an uncertain consequence (Jagadeesh, et al., Nature Genetics 2016, 48(12), 1581).

In the case of simulating a population of offspring, RTG tool was used for generating simulated children. However, RTG tool does not consider the linkage disequilibrium process. Linkage disequilibrium can be incorporated into the child genome generation process (using, e.g., the RTG tool) by using the variable/non-random recombination rates that occur in real human populations. This can be accomplished using any suitable recombination rate data. For example, recombination rate data can be obtained by analyzing the recombination rates and/or linkage disequilibrium observed and/or measured in real human populations. Such recombination maps have already been generated and such recombination rate maps can be used in the disclosed methods. For example, recombination rate maps for human genome build 37 (GRCh37) (Bhérer, et al., Nature Communications 2017, 8, 14994) were used herein to determine recombination probabilities. Preferably, such recombination rate maps provide a thorough analysis of the variation in recombination rate between females and males (such a thorough analysis is provided by GRCh37). The GRCh37 maps were downloaded from the NCBI web resource (web site ncbi.nlm.nih.gov/assembly). GRCh37 is derived from 3.3 million crossovers from 104,246 meioses (57,919 female and 46,327 male meioses) (https://www.ncbi.nlm nih.gov/assembly/GCF_000001405.134

Since the recombination probability can be used to determine the number of recombinations per chromosome, the recombination rate Map(cM) was converted to recombination probability using Formula (1), following methods described in Su, et al., Science 1999, 286(5443), 1351-1353.

$$Pr [ recombination \text{\textbar} linkage of d cM ] = {\sum }_{k = 0}^{∞} Pr [ 2 k + 1 crossovers \text{\textbar} linkage of d cM ] = {\sum }_{k = 0}^{∞} exp ( - d / 100 ) \frac{{( d / 100 )}^{2 k + 1}}{( 2 k + 1 ) !} = exp ( - d / 100 ) sinh ( d / 100 ) = \frac{1 - exp ( - 2 d / 100 )}{2} . \qquad ( 1 )$$

Formula 1 provides a recombination probability for each chromosomal position in the human genome. This probability distribution was utilized to draw n or m times (n and m are parameters that determine the number of cross-overs per chromosome in males and females, respectively) from each chromosome to decide the location of a crossover.

With knowledge of the recombination probability, the number of crossovers required can be readily determined. For example, for a chromosome of length 100, and for two recombinations, one iterates through all 100 chromosomal positions and decides for each whether to recombine or not. As a result, there is on average two recombinations.

The simulator takes two VCF files as input (representing the genotype information of a mother and a father). Then, the algorithm combines them into a single VCF file. After combining the VCF files, the simulator generates a population of simulated children (the default number is 100) taking into account the recombination probabilities as described herein. After this step, one can follow the same procedures for annotating and analyzing individual variants for the resulting simulated offspring genome, in terms of predicting the pathogenic variants for all the associated diseases from the databases. Then, a statistical summary (likelihoods) for all the diseases associated with the population of offspring is generated, i.e., how many individuals in the simulated cohort are carrying certain disease-associated variants. Finally, a new file is generated, containing annotations of all the related information in a format that can be visualized. The summary statistics (and individuals within the simulated cohort) can then be visualized similarly to the visualization of individual VCF files. Algorithm 1 illustrates the procedure that was followed for the simulation. FIGS. 1A and 1B provide overviews of the overall workflow.

Algorithm 1: Simulations Algorithm

Input : - Two VCF files represent Father and Mother genomics data.

- Genetic maps for male and female.

Output: Summary statistics of different diseases and symptoms associated with offspring
START: - Merge the two input file using VCFtool.

- Compute recombination probability for each genomic position using recombination

maps

| | - (N [symbol] M) ← (mother [symbol] father) crossover locations | | |
|---|---|---|---|
| | for i := 1 → NumberOfChildren do | | |
| \| | for i:= 1 → NumberOfVariantes do | | |
| \| | \| | | if VariantsPosi [symbol] (N∥M) then |
| \| | \| | \| | Perform the crossover in the selected recombination positions [symbol] |
| \| | \| | | else |
| \| | \| | \|_ | Go to the next position : |
| \| | \|_ | | create : Child; |

| | | |
|---|---|---|
| \| | 1 | - Matches with ClinVar DB for Mendelian Diseases Variants |
| \| | 2 | - Matches with GWAS DB for Complex Disease Variants |
| \| | 3 | - Matches with DIDA DB for Digenic Disease Variants |
| \| | 4 | - Matches with PharmGKB DB for Drug Response |
| \| | n | - Predicted as Pathogenic Relevant Variants using MCAP |
| \| | | Result : (VCF file) ← Combine results and generate counts and summary |
| \|_ | | statistics |
| | | return (VCFfile with Summary Statistics); |

[symbol]

indicates data missing or illegible when filed

### Results

VSIM is web-based simulation and visualization tool that aims to support genetic counseling and interpretation of data associated with genomic sequences. VSIM performs two main operations: First: VSIM is able to annotate and visualize personal genomes available in the VCF file format (Danecek, et al., Bioinformatics 2011, 27(15), 2156-2158) in order to support visual exploration of variants and other genomic aberrations that may have an impact on health. The VCF file contains variations such as SNP (single nucleotide polymorphism) and InDel (insertion and deletion) for one individual. VSIM identifies the candidate disease variants by referencing to different databases.

Second: given two VCF files for two potential parents, VSIM can simulate a population of children, based on accurately accounting for recombination probabilities across the human genome, and then allows visual exploration of the simulation results. One of the main applications of the second feature of VSIM is genetic counseling and premarital genetic testing. However, the simulation and annotation of genomes can also be used for evolutionary studies.

A. Annotating and Visualizing Personal Genomic Data VSIM accepts a VCF file as input, annotates the variants in the VCF file, and visualizes the results on a chromosomal ideogram. The VCF file at a minimum must include the chromosome number (#CHROM), position (POS), reference alleles (REF) and alternate alleles (ALT), and information (INFO). Then, the variants in VCF files are annotated with different information. The variants were annotated with information related to the databases shown in Table 1. Annotation of variants falls into four or five categories: known Mendelian disease variants using information from the Clinvar database (Landrum, et al., Nucleic Acids Research 2013, 42(D1) D980-D985); disease-associated variants derived from GWAS studies using information from the GWAS catalog (MacArthur, et al., Nucleic Acids Research 2016, 45(D1), D896-D901) for Complex Diseases; variant combinations in di-genic disease using information provided by the DIDA database (Gazzo, Nucleic Acids Research 2015, 44(D1), D900-D907); and pharmacogenomic variants from the PharmGKB database (Jagadeesh, et al., Nature Genetics 2016, 48(12), 1581) for the Drug response. A fifth category involves predicted pathogenic variants using the M-CAP pathogenicity score (Jagadeesh, et al., Nature Genetics 2016, 48(12), 1581) which is a pathogenicity classifier for the rare missense variants in the human genome that are tuned to the high sensitivity required in the clinic. This method of prediction is used to score all the variants and predict the pathogenic one.

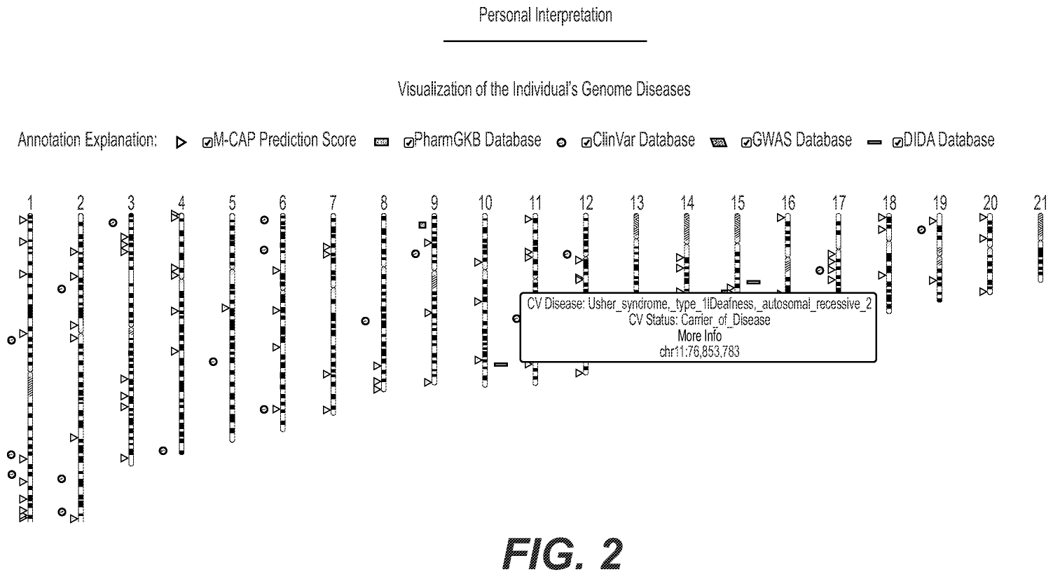

VSIM then generates chromosomal views based on chromosomal ideograms and shows the chromosomal positions at which functional variants have been found. This chromosome-focused visualization facilitates, for example, identifying haplotype blocks that are enriched for functional variants. Different categories of variants are shown in different colors, and it is possible to filter variants by their type (e.g., whether they are Mendelian disease variants, pharmacogenomic variants, etc.). Users are able to obtain additional information about variants when selecting a single variant, and can follow a hyper-link to a website with additional information and evidence about the type of variant. FIG. 2 provides an example of the visual output produced by VSIM from a single VCF file.

B. Simulating Child Cohorts and Application to Premarital Testing

VSIM is further capable of simulating cohorts of potential child genomes when given two VCF files as input, and using this simulated cohort to estimate the probability of encountering particular genetically based diseases in potential children (as well as the co-morbidities between the diseases). For this purpose, VSIM uses a map of genome-wide recombination rates for the human genome (Bhérer, et al., Nature Communications 2017, 8, 14994), which provides a global (i.e., not population-specific) estimate of recombination rates, distinguished by male and female genomes. VSIM tool investigates potential disease outcomes during premarital genetic screening, by simulating a population of potential children, analyzing diseases that might be present or carried based on the genetic factors of their parents, and presenting the results in a visual format. The simulation algorithm is based on the RTG simulation tool (Cleary, BioRxiv 2015, p. 023754). This tool provides a blueprint platform for genomic analysis. RTG tools software is available as an executable file with multiple commands executed through a command line interface. However, RTG simulation does not have any capability to simulate populations while maintaining linkage disequilibrium. Therefore, the RTG method has been updated to capture the linkage disequilibrium. Recombination rate maps for human genome build 37 were used, and were relied on for an analysis of the variation in recombination rate between females and males derived from 3.3 million crossovers from 104,246 meioses (57,919 female and 46,327 male meioses) (Bhérer, et al., Nature Communications 2017, 8, 14994). Then, the recombination probability is calculated, which helps to determine the number of the crossovers required per chromosome. After that, based on the recombination probability a cumulative distribution function (CDF) was calculated, from which crossover positions were obtained.

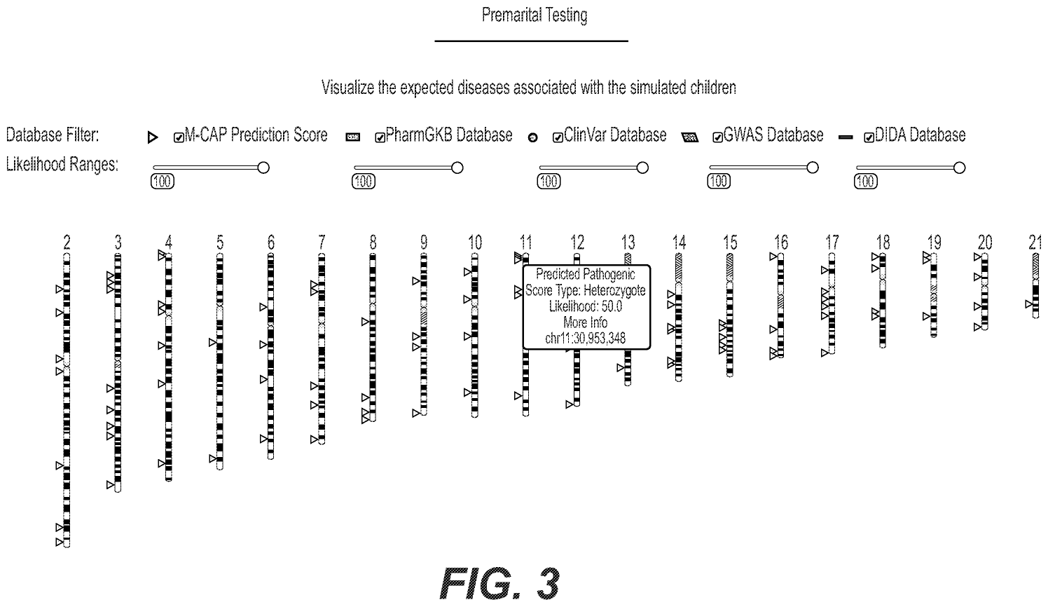

Using two input VCF files, the recombination rates and a parameter that determines the number of cross-overs per chromosome, VSIM simulates a population of potential children while considering the recombination probabilities; therefore, the population of children will account for, at least partially, linkage disequilibrium and the resulting correlation between risk-conveying or causative genomic positions. All genomes in the simulated cohort of children were annotated using the same annotation procedure and annotation sources used by VSIM. The percentage of children within the population that carries a particular functional variant was used to estimate the likelihood that children will develop or carry a particular disease. While the likelihood could be estimated directly using Mendel's laws from the two parent genomes in Mendelian diseases, this simulation approach will give more accurate probabilities in the case of complex, digenic, and oligogenic diseases, and will further allow the estimation of co-morbidities in the child population. FIG. 3 provides an example of the simulation result and its visualization. The simulator requires two VCF files as an (representing the mother and father genotype information). Then, the algorithm combines them into one VCF file. After that, it generates simulated children (the default number is 100). The algorithm then follows the same procedures for analyzing individual variants, in terms of predicting pathogenic variants for all of the associated diseases from the databases. Then, statistical summary (likelihoods) for all the diseases associated with children is generated. Finally, it creates a new file annotated with all the related information in a format that can be visualized.

C. Performance Evaluation

Annotation of individual variants is relatively fast. However, the time it takes to analyze (i.e., annotate and visualize) a single whole genome depends on the size of the VCF file. For a VCF with three million SNPs, VSIM takes approximately 10 minutes to generate the final output using an Intel i7 processor at 2.5 GHz with 16 GB of memory. VSIM annotates a single variant on average in 1.4×10⁻⁴ seconds. When applying VSIM for determining the likelihood of children having or carrying a particular disease, the simulation time not only depends on the size of the VCF file, it also depends on the number of simulated children. FIG. 4 shows the performance benchmarks for different numbers of simulated children. As shown, the time increases linearly with the number of simulations to perform. Therefore, the generation of simulated genomes can easily be parallelized.

In addition to time, the number of generations that the simulator needs to produce linkage disequilibrium as observed in a real population was evaluated. Starting with a randomly generated population of individuals, a two individuals in this population are randomly paired to generate a single child genome. This pairing of individuals and child genome generation is repeated until a certain number of child genomes have been generated for this population. Then, the simulation moves forward one generation and repeats this process, using the child genomes of the previous generation as the population from which individuals are randomly paired to generate child genomes for the new generation. After each generation, the linkage disequilibrium is measured and compared to the linkage disequilibrium in a human population used to generate the linkage maps. In the initial population having only completely random genomes there is no linkage disequilibrium that resembles a real population. The simulation algorithm then introduces linkage disequilibrium due to random but non-uniformly distributed recombination events so that after several generations the linkage disequilibrium should approximate the linkage found in a human population. FIG. 5 shows the correlation value for the first seven generations. The correlation increases from one generation to the next in succession, and a strong correlation with linkage disequilibrium in a human population emerges after only a few generations. Having demonstrated that VSIM can create linkage disequilibrium starting from a random population, the aspect of generating several cycles of child genome is an optional feature of VSIM.

VSIM is an automated and easy to use web application for interpretation and visualization of a variety of genomics data, in particular interpretation of individual genomes. Underlying VSIM is a genome simulation algorithm that accounts for non-uniformly distributed recombination rates and can be used to create linkage disequilibrium in simulated populations. VSIM can use this simulator to help predict, and to provide a general overview of the potential diseases that might be associated with children. While this approach is applicable to any disease, it is particularly relevant with diseases that are associated with more than one genomic locus. For premarital testing, VSIM has several limitations, including the limited number of databases for annotation of genomic variants, its lack of consideration for X-or Y-linked phenotypes, and limited number of polygenic sites and risk scores (mainly coming from known GWAS studies). In the future, VSIM can be extended with additional information about effect sizes of variants and combinations of variants in particular for oligogenic and polygenic disease.

VSIM identifies the candidate diseases variants by referencing to four databases Clinvar, GWAS, DIDA, and PharmGKB, and predicted the pathogenic variants. Moreover, it investigates the attitude towards premarital genetic screening by simulation number of children and analysis the diseases that might be carrying or have, based on the genetic factors of their parents and visualize the result. VSIM supports output formats that easy to interpret and understand, which makes it a biologist-friendly powerful tool for data visualization and interpretation. VSIM can be applied in clinical environments for visual interpretation of whole exome or whole genome sequences of individuals.

Further, the simulator underlying VSIM can also be used as a tool for the study of genetic associations of diseases as well as correlation between different disease-associated loci and their progression within a population. Its application, therefore, goes beyond premarital testing or interpretation of genomics.

Those skilled in the art will recognize, or be able to ascertain using no more than routine experimentation, many equivalents to the specific embodiments of the invention described herein. Such equivalents are intended to be encompassed by the following claims.

1. A computer-implemented method (CIM) for analyzing genomic data, the CIM comprising:
(a) generating a population of offspring genomes from the genomes of the two individuals, taking into account linkage disequilibrium; and
(b) visualizing a probability of morbidity associated with the population of offspring genomes.

2. The CIM of claim 1, wherein the linkage disequilibrium taken into account comprises the linkage disequilibrium found in a human population.

3. The CIM of claim 1, wherein the genomes of the two individuals are provided in a file format selected from the group consisting of a Variant Call Format (VCF), a Genome Variation Format (GVF), a Generic Feature Format (GFF), Gene Transfer Format (GTF), Tab Separated File (TSV), annovar file format, and masterVar file format, preferably VCF or GFF.

4. The CIM of claim 1, further comprising combining the genomes of the two individuals into a single file prior to step (a).

5. The CIM of claim 1, further comprising a step of (i) annotating each genome in the population of offspring genomes with disease variants from one or more genomic databases after step (a) and prior to step (b).

6. The CIM of claim 1, further comprising a step of (ii) predicting pathogenic variants in each genome in the population of offspring genomes after step (i) and prior to step (b).

7. The CIM of claim 5, further comprising a step of (iii) performing a statistical analysis to determine the probability of morbidity after step (ii) and prior to step (b).

8. The CIM of claim 6, wherein predicting pathogenic variants is performed using a Mendelian Clinically Applicable Pathogenicity (M-CAP) score, ClinPred, xgboost, cforest, VESTS, MetaSVM, REVEL, MetaLR, Eigen, GenoCanyon, REVEL, Fathmm_MKL, SIFT, Polyphen-2, or CADD, preferably M-CAP.

9. The CIM of claim 1, wherein taking into account linkage disequilibrium comprises using recombination probabilities and a parameter that determines number of cross-overs per chromosome.

10. The CIM of claim 9, wherein the recombination probabilities are determined using a set of precomputed rate maps for a human genome build such as human genome build 37 or later versions such as GRCh38 and GRCh39.

11. The CIM of claim 5, wherein the one or more genomic databases are selected from the group consisting of ClinVar database, Genome-Wide Association Studies (GWAS) database, DIgenic disease DAtabase (DIDA), Pharmacogenomics Knowledgebase (PharmGKB), and combinations thereof.

12. The CIM of claim 5, wherein the one or more genomic databases are the ClinVar database, GWAS database, DIDA, and PharmGKB.

13. The CIM of claim 5, wherein the one or more genomic databases comprise a database containing information about Mendelian diseases; genetic associations for risk factors and/or complex diseases variants; digenic disease variants; oligogenic disease variants; pharmacogenomic variants; lifestyle factors; environmental factors; or a combination thereof.

14. The CIM of claim 5, wherein the one or more genomic databases comprise a database containing information about complex diseases variants, digenic disease variants, oligogenic disease variants, or a combination thereof.

15. The CIM of claim 5, wherein the one or more genomic databases are dynamic.

16. The CIM of claim 5, where the one or more genomic databases are stored on one or more hardware modules.

17. The CIM of claim 1, wherein the genomes of the two individuals are provided to a first user interface hardware module, such as a graphical user interface (such as a digital screen), configured to receive genome from at least one of the two individuals, preferably wherein the first user interface hardware module is operably linked to the one or more hardware modules via ethernet, bluetooth, near field communication, WiFi, integrated circuits, or a combination thereof.

18. The CIM of 17, wherein visualizing the probability of morbidity occurs on the first user interface hardware module, a second user interface hardware module such as a graphical user interface (such as a digital screen), or both, preferably wherein the second user interface hardware module is operably linked to the one or more hardware modules ethernet, bluetooth, near field communication, WiFi, integrated circuits, or a combination thereof.

19. The CIM of claim 18, wherein the second user interface hardware module is operably linked to the one or more hardware modules via ethernet, bluetooth, near field communication, WiFi, integrated circuits, or a combination thereof.

20. The CIM of claim 17, wherein generating the population of offspring genomes occurs on a third hardware module, preferably wherein the third hardware module is operably linked to:
the first user interface hardware module;
the second user interface hardware module; and/or
the one or more hardware modules.

21. The CIM of claim 18, wherein the first user interface hardware module or the second user interface hardware module and the third hardware module are on the same device or on different devices.

22. The CIM of claim 1, further comprising a step of utilizing the probability of morbidity to counsel at least one of the two individuals.

23. A computer-implemented system (CIS) for analyzing gene expression data, comprising an informatics tool that generates a population of offspring genomes from the genomic information taking into account linkage disequilibrium, and provides processed results to a user.

24. The CIS of claim 23, further comprising a user interface hardware module, such as a graphical user interface (such as a digital screen), configured to receive genomic information from the user or another user.

25. The CIS of claim 23, wherein the CIS allows for implementation of a CIM comprising:
(a) generating a population of offspring genomes from the genomes of the two individuals, taking into account linkage disequilibrium; and
(b) visualizing a probability of morbidity associated with the population of offspring genomes.
